(19) United States
(12) Patent Application Publication
(10) Pub. No.: US 2009/0012013 A9
(43) Pub. Date: Jan. 8, 2009

(54) Steroid modified solatrioses

(76) Inventors: Christopher John Lawson, Reading (GB); Alexander Charles Weymouth-Wilson, Reading (GB)

Correspondence Address: KAGAN BINDER, PLLC, SUITE 200, MAPLE ISLAND BUILDING, 221 MAIN STREET NORTH, STILLWATER, MN, 55082, US

(21) Appl. No.: 10/563,745

(22) PCT Filed: Jul. 8, 2004
(86) PCT No.: PCT/EP04/07538

§ 371 Date: Aug. 29, 2006

(30) Foreign Application Priority Data
Jul. 8, 2003 (EP) 03015501.4

(51) Int. Cl. A61K 31/704; C07J 17/00

(52) U.S. Cl. 514/26; 536/6.1

(57) ABSTRACT

The invention pertains to steroid modified solatrioses and the synthesis thereof as well as to intermediate compounds useful for the synthesis of the steroid modified solatrioses.

The present invention relates to the chemical synthesis of alkaloid glycosides, in particular to the synthesis of steroid modified solatrioses. Furthermore, the present invention relates to novel steroid modified solatrioses and intermediate compounds useful for the synthesis thereof.

Solasodine and its glycosides are of considerable interest commercially and clinically. They are widely used as starting products for the synthesis of various steroidal drugs. The aglycon solasodine is a source for synthetic cortisone and progesterone.

It is moreover well established that certain naturally occurring conjugate solasodine glycosides have potent antineoplastic properties. Of particular interest is the triglycoside solasonine (22R, 25R)-spiro-5-en-3β-yl-α-L-rhamno-pyranosyl-(1->2 gal)-O-p-D-glucopyranosyl-(1->3 gal)-β-D-galactopyranose. The structure of this triglycoside is as follows:

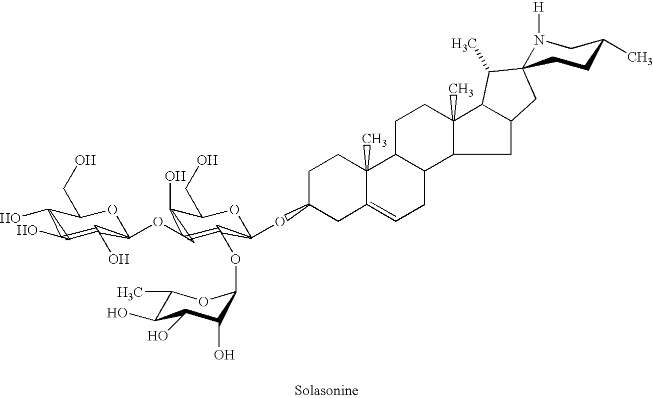

The above triglycoside is conventionally obtained by extraction from a plant source. A commercially available extract of S. sodomaeum, commonly referred to as BEC (Drug Future, 1988, vol. 13.8, pages 714-716) is a crude mixture of solamargine, solasonine and their isomeric diglycosides. The extraction process for making BEC involves homogenizing the fruits of S. sodomaeum in a large volume of acetic acid, filtering off the liquid through muslin followed by precipitation of the glycosides with ammonia (Drugs of today (1990), Vol. 26 No. 1, p. 55-58, cancer letters (1991), Vol. 59, p. 183-192). The yield of the solasodine glycoside mixture is very low (approx. 1%). Moreover the individual process steps are not defined to GMP in terms of scale up, definition of yield, composition and product quality.

There is a great need for a cost efficient process that provides the antineoplastically active triglycoside solasonine at high yield with little or no impurities. Contrary to other steroid ring systems, the steroid skeleton of solasodine contains a very labile nitrogen-containing ring. The same holds true for the steroid ring systems of relared alkaloids such as tomatidine, demissidine or solanidine. These aglycons cannot readily be chemically modified while keeping the steroid skeleton intact. In spite of the fact that the aglycon solasodine is readily available, the prior art does not disclose the synthesis of the solasonine using the aglycon material as starting material.

The synthesis of solasonine requires the stereoselective glycosylation of solasodine at the relatively unreactive hydroxyl group.

It has been found that solasodine is not compatible with the conventional steroid glycosylation technique. No glycosylation was observed following the treatment of solasodine with tetrabenzoyl α-D-glucopyranosyl trichloroacetimidate and trimethyl-silyl triflate or boron trifluoride dietherate (unpublished results).

The problem underlying the present invention is to provide a cost effective method for the preparation of solasonine and solasonine analogues in high yields.

Such compounds exhibit cytotoxic activity and may be employed as anticancer agents. Furthermore, such compounds exhibit anti bacterial, anti fungal or anti viral activity.

Accordingly, the present invention provides a method for the preparation of a steroid modified solatriose of general formula (I): 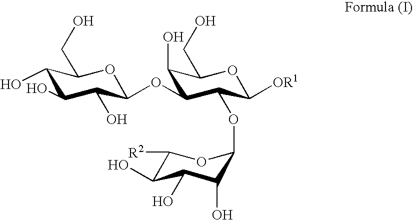
wherein R¹ represents a steroid or a derivative thereof having a hydroxyl group in 3-position and no further unprotected hydroxyl groups; and R² represents a straight or branched C₁₋₄ alkyl group or a hydroxyl group.

The method of the present invention comprises the step of: reacting a compound of general formula (XIII): 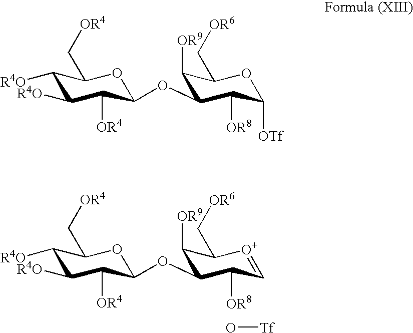
wherein each R⁴ independently represents a benzoyl, acetyl or pivolyl protecting group; R⁶ represents a pivolyl protecting group; R⁸ represents a chloroacetyl protecting group; R⁹ represents a benzoyl, acetyl or pivolyl protecting group and Tf represents a triflate leaving group; with a compound of general formula (XIV):
HO—R¹ Formula (XIV)
wherein R¹ is as defined above,

to yield a compound of general formula (XV): 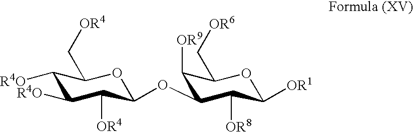
wherein R¹, R⁶, R⁸ and R⁹ are as defined above.

The compound of the above general formula (XV) may be transformed to the desired steroid modified solatriose of general formula (I) by any suitable method known in the art. A particular preferred procedure is described in detail below.

Furthermore, the present application provides steroid modified solatriose compounds of general formula (I) as defined above, wherein R¹ represents a tomatidin-3-yl, demissidin-3-yl, solanidin-3-yl or solasodin-3-yl group.

A further object of the present application is the provision of intermediate compounds useful for the synthesis of the steroid modified solatriose of general formula (I) defined above, namely:

A compound of general formula (XVII): 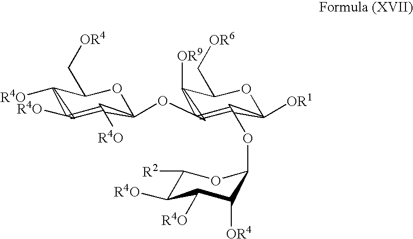
wherein R¹, R², R⁴, R⁶, and R⁹ are as defined above.

A compound of general formula (XV) as defined above

A compound of general formula (X): 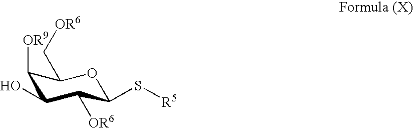
wherein R⁶, R⁸ and R⁹ are as defined above; and R⁵ represents a straight or branched C₁₋₁₄ alkyl group or a phenyl group optionally substituted with one or more C₁₋₄ alkyl groups, halogen atom such as Cl, F, Br or 1, or N0₂ group. A compound of general formula (XII): 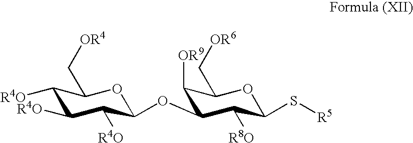
wherein R⁴, R⁵, R⁶, R⁸ and R⁹ are as defined above.

Further embodiments of the present application are described in the dependent claims.

### DETAILED DESCRIPTION OF THE INVENTION

In the following, the present invention will be explained in more detail with reference to preferred embodiments.

The steroid residue constituting substituent R¹ is a steroid or a derivative thereof having a hydroxyl group in the 3-position for bonding as α-glycosidic hydroxyl group in the compound of general formula (I). The steroid residue bears no further unprotected hydroxyl groups and preferably has no further hydroxyl groups at all, in order not to compromise subsequent reaction steps. In a preferred embodiment of the present invention R¹ is selected from a tomatidin-3-yl, demissidin-3-yl, solanidin-3-yl and solasodin-3-yl group.

All of those steroid groups contain a labile nitrogen-containing ring and, therefore, cannot be chemically modified by means of conventional methods. Moreover, all of the above steroid groups represent substituents for cyctotoxic, anti bacterial, anti fungal or anti viral compounds.

In the above general formula (I) each R² independently represents a straight or branched alkyl group having 1 to 4 carbon atoms or a hydroxyl group. In a preferred embodiment, R² represents a methyl group.

According to a preferred embodiment of the method of the present invention, galactose is reacted in step (A) to yield a compound of general formula (II ): 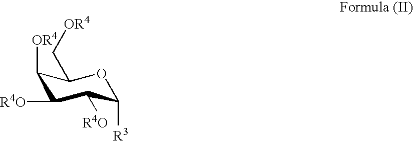
wherein R³ represents a chlorine or bromine atom; and each R⁴ independently represents a benzoyl, acetyl or pivolyl protecting group. In a preferred embodiment R³ represent a bromine atom. In another preferred embodiment R⁴ represents an acetyl protecting group. Step (A) may be carried out using either acetic anhydride, acetyl chloride, benzoyl chloride, benzoic anhydride, or pivolyl chloride in the presence of a base such as, e.g., pyridine, triethylamine, or collidine, to give fully esterified galactose. Esterified-D-galactopyranose may be treated with hydrogenbromide or hydrogenchloride in glacial acetic acid to yield the above compound of general formula (II).

In a particularly preferred embodiment galactose is suspended in organic base such as pyridine and cooled to 0° C., to this solution is added dropwise either acetic anhydride, benzoic anhydride or acid chloride. Upon complete addition the solution is warmed to +25° C. (room temperature) and stirred for about 16 hours. The reaction is quenched by addition of alcohol. The solution is diluted with organic solvent such as tert-butylmethyl ether, or dichloromethane, or toluene and washed with cold 1N HCl, water, saturated sodium bicarbonate, water and brine then the product is dried over magnesium sulfate and concentrated under reduced pressure to dryness. The product can be used without further purification or it can be recrystallised. The fully esterified galactopyranose in dry solvent such as dichloromethane is cooled to 0° C. under an inert atmosphere. To this solution is added hydrogen bromide in glacial acetic acid, typically 30% HBr content. The solution is allowed to warm to +25° C. (room temperature) and stirred for around 16 hours. The solution is diluted with organic solvent such as dichloromethane and then quickly washed with ice cold water, saturated aqueous sodium bicarbonate, and brine. The product is dried over magnesium sulfate filtered and the solvent is removed under reduced pressure. The product is crystallized from petrol (40-60) and diethyl ether.

In step (B), a compound of general formula (II) is reacted with a compound of general formula (III):
HS—R⁵ Formula (III)
wherein R⁵ represents a straight or branched C₁₋₁₄ alkyl group or a phenyl group optionally substituted with one or more C₁₋₄ alkyl groups; whereby the C1-14 alkyl groups are preferably selected from methyl, ethyl and propyl and the phenyl group is preferably selected form phenyl, p-methylphenyl and p-chlorophenyl; and methyl, ethyl and propyl are particularly preferred; to yield a compound of general formula (IV): 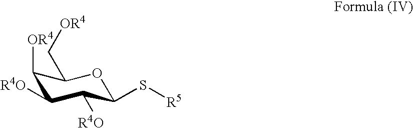
wherein R⁴ and R⁵ are as defined above.

Preferably R⁵ is a phenyl group.

Furthermore, in step (C), the compound of general formula (IV) is deprotected to yield a compound of general formula (V): 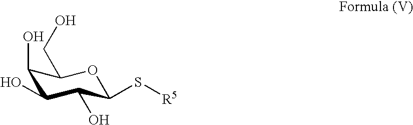
wherein R⁵ is as defined above.

Any suitable deprotection condition conventionally employed in the chemistry of protecting groups may be used. Deprotection is preferably be carried out in an inert organic solvent such as dichloromethane or tetrahydrofuran in the presence of an alkali metal alkoxide having 1 to 4 carbon atoms and a C₁₋₄ alcohol, or in the presence of water, an alkali metal hydroxide and a C₁₋₄ alcohol. In a particular preferred embodiment deprotection in step (C) is carried out in dry methanol with catalytic amount of sodium methoxide.

Subsequently, the OH group in 6-position is selectively protected in step (D) using a bulky protecting group to yield a compound of general formula (VI) 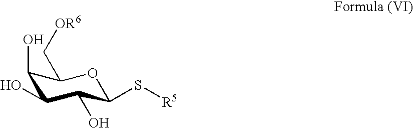
wherein R⁵ is as defined above; and R⁶ is a pivolyl, benzoyl or substituted benzoyl protecting group, whereby the substituents are selected from alkyl groups such as methyl, halogen atoms such as Cl, Br, F, and I and NO₂. Preferably R⁶ represents a pivolyl protecting group.

In a preferred embodiment the reaction may be carried out using pivolyl chloride in dry dichloromethane in the presence of pyridine.

In step (E), the OH groups in 3- and 4-position are selectively protected with a ketal or acetal protecting group using standard conditions to yield a compound of general formula (VII): 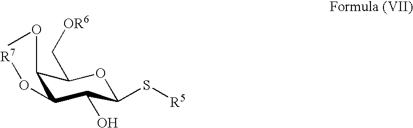
wherein R⁵ and R⁶ are as defined above; and R⁷ represents a ketal or acetal type protecting group selected from benzylidene, 4-nitrobenzylidene, 4-methoxybenzylidene or isopropylidene. In a preferred embodiment R⁷ represents an isopropylidene protecting group.

The reaction is preferably carried out in a dipolar aprotic solvent such as dimethyl formamide (DMF) or acetone in the presence of acid catalysts such as p-toluene sulfonic acid or camphorsulfonic acid using a 2,2-dialkyloxypropane or an optionally substituted dialkylbenzylidene.

Suitable reaction temperatures range from ambient temperature to elevated temperatures. Preferably the reaction is carried out at a temperature of 25° C.

Moreover, the OH group in 2-position is protected in step (F) by reacting the compound of general formula (VII) with chloroacetyl chloride to yield a compound of general formula (VIII): 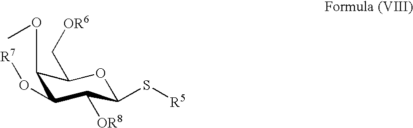
wherein R⁵, R⁷ and R⁸ are as defined above; and R⁸ represents a chloroacetyl protecting group.

The reaction may be carried out in a dry solvent such as dichloromethane with a base such as pyridine or triethylamine at a temperature of from 0° C. to 25° C.

In step (G) the compound of general formula (VIII) is deprotected to yield a compound of general formula (IX): 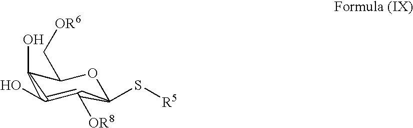
wherein R⁵, R⁶ and R⁸ are as defined above.

Deprotection may be carried out under acidic conditions by treating with aqueous acetic acid, aqueous trifluoroacetic acid or mineral or sulfonic acid.

In step (H) the compound of general formula (IX) is reacted with a trialkylorthoacetate, benzoate or pivolate, wherein the alkyl residues have 1 to 4 carbon atoms, to form an 3,4-ortho ester which is subsequently migrated to the axial 4-position under acidic conditions to yield a compound of general formula (X): 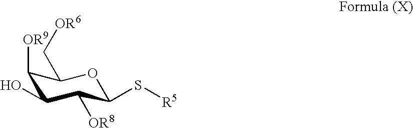
wherein R⁵, R⁶, R⁸ are as defined above and R⁹ is an acetyl, benzoyl or pivolyl protecting group. In preferred embodiments R⁹ represent an acetate or benzoyl protecting group, which may be introduced by means of trimethyl or triethyl orthoacetate or benzoate, most preferably trimethylorthoacetate.

Step (H) may be conducted in an inert organic solvent such as acetonitrile.

Preferably the reaction is carried out in the presence of a catalyst. Any conventional catalyst used in carbohydrate chemistry may be employed. Particular preferred catalysts include p-toluenesulfonic acid, or camphor sulfonic acid. The most preferred catalyst is p-toluenesulfonic acid.

The reaction may preferably be carried out under anhydrous conditions in the presence of a water detracting means such as 4 Å mol sieves.

The free OH group in 3-position is reacted in step (I) with a protected halogen glucose derivative of general formula (XI): 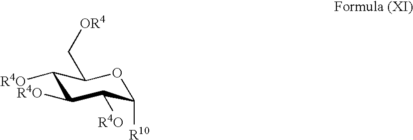
wherein R⁴ is as defined above; and R¹⁰ represent a halogen atom such as fluorine, chlorine or bromine, to yield a compound of general formula (XII): 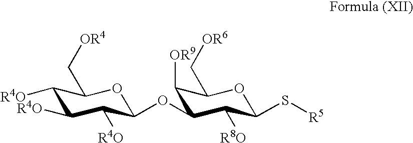
wherein R⁴, R⁵, R, R⁸ and R⁹ are as defined above.

The reaction is preferably carried out in the presence of promoters such as silver triflate, zinc dichloride, borontrifluoride diethyletherate, or N-iodosuccinamide/triflic acid.

In a preferred embodiment a dry solvent such as dichloromethane is employed. The reaction temperature is preferably at a range of from −20° C. to 25° C.

Activating compound (XII) may be achived in step (J) through the oxidiation of the thio ether to the sulfoxide and the formation of the anomer trifate of general formula (XIII) below, which may exist as either the alpha triflate or the alpha ion pair: 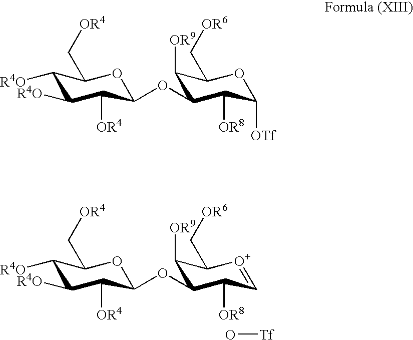
wherein R⁴, R⁵, R⁶, R⁸ and R⁹ are as defined above.

The reaction is preferably carried out by oxidizing the thio ether group to a sulfoxide using hydrogen peroxide, and subsequently treating the resulting intermediate with triflic anhydride. Furthermore, in a particular preferred embodiment, a sterically hindered non-nucleophilic base such as 2,6-lutidine, 2,4,6-collidine or 2,6-di-tertbutyl-4-methyl-pyridine is present. The most preferred sterically hindered base is 2,6-di-tertbutyl-4-methly-pyridine.

In step (K), coupling of the compound of general formula (XIII) with the compound of general formula (XIV)
HO—R¹ Formula (XIV)
wherein R¹ is as defined above; may be performed in the presence of sterically hindered non-nucleophilic base such as 2,6-lutidine, 2,4,6-collidine or 2,6-di-tertbutyl-4-methyl pyridine, preferably 2,6-di-tertbutyl-4-methyl-pyridine, to yield a compound of general formula (XV): 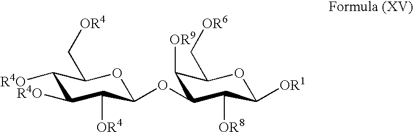
wherein R¹, R⁶, R⁸ and R⁹ are as defined above.

The reaction may preferably be carried out under anhydrous conditions in the presence of a water detracting means such as 4 Å mol sieves.

In a preferred embodiment the reaction is carried out at low temperature such as 0° C. or lower, more preferably −10° C. or lower. The most preferred reaction temperature is −20° C.

In step (L), the OH group in 2-position substituted with R⁸ is selectively deprotected using thio urea in the presence of a sterically hindered base such as 2,6-lutidine, 2,4,6-collidine or 2,6-di-tertbutyl-4-methyl pyridine, preferably 2,6-lutidine, in a dry alcohol such as methanol, ethanol or isopropanol, preferably ethanol, and subsequently reacted with a protected halogen rhanmose derivative of general formula (XVI): 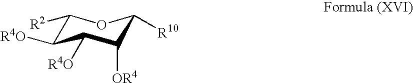
wherein R² and R⁴ are as defined above; and R¹¹ represents a halogen atom such as bromine, chlorine or fluorine, preferably bromine, to yield a compound of general formula (XVII): 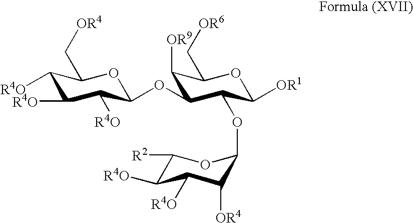
wherein R¹, R², R⁴, R⁶, and R⁹ are as defined above.

The deprotection in step (M) may be performed under substantially the same conditions as described above for step (C) to yield the compound of general formula (I). In a preferred embodiment, deesterification may by accomplished using sodium methoxide in a methanol/dichloromethane mixture.

1. A method for the preparation of a steroid modified solatriose of general formula (I): 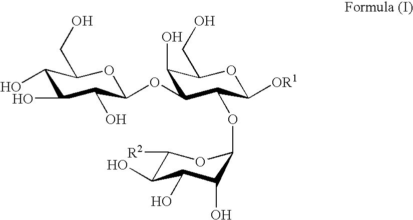
wherein R¹ represents a steroid or a derivative thereof having a hydroxyl group in 3-position and no further unprotected hydroxyl groups; and R² represents a straight or branched C₁₋₄ alkyl group or a hydroxyl group, which method comprises the step of:
reacting a compound of general formula (XIII): 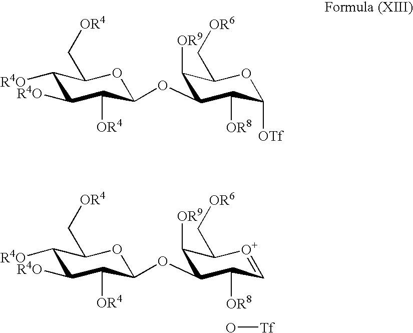
wherein each R⁴ independently represents a benzoyl, acetyl or pivolyl protecting group; R⁶ represents a pivolyl protecting group; R⁸ represents a chloroacetyl protecting group; R⁹ represents a benzoyl, acetyl or pivolyl protecting group; and Tf represents a triflate leaving group;
with a compound of general formula (XIV):
HO—R¹ Formula (XIV)
wherein R¹ is as defined above
to yield a compound of general formula (XV): 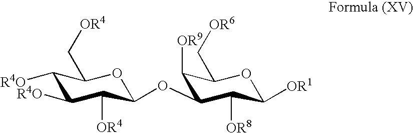
wherein R¹, R⁶, R⁸ and R⁹ are as defined above.

2. The method according to claim 1, further comprising the step of: reacting galactose to yield a galactose fully protected with ester type protecting groups, and subsequently treating with hydrogen bromide or hydrogen chloride to yield a compound of general formula (II): 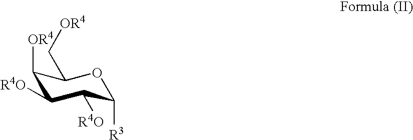
wherein R³ represents a chlorine or bromine atom; and R⁴ is as defined in claim 1.

3. The method according to claim 2, further comprising the step:
reacting a compound of general formula (II) as defined in claim 2, with a compound of general formula (III):
HS—R⁵ Formula (III)
wherein R⁵ represents a straight or branched C₁₋₁₄ alkyl group or a phenyl group optionally substituted with one or more C₁₋₄ alkyl groups;
to yield a compound of general formula (IV): 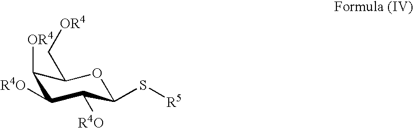
wherein R⁴ independently represents a benzoyl, acetyl or pivolyl protecting group, and R⁵ is as defined above.

4. The method according to claim 3, further comprising the step of:
deprotecting a compound of general formula (IV) as defined in claim 3 to yield a compound of general formula (V): 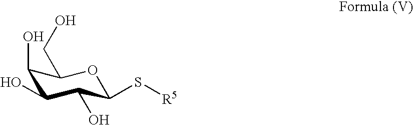
wherein R⁵ is as defined in claim 3.

5. The method according to claim 4, further comprising the step of:
selectively protecting the OH group in the 6-position of a compound of formula (V) as defined in claim 4 with pivolyl chloride using standard conditions to yield a compound of general formula (VI): 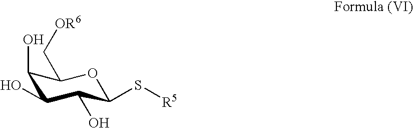
wherein R⁵ represents a straight or branched C₁₋₁₄ alkyl group or a phenyl group optionally substituted with one or more C₁₋₄ alkyl groups; and R⁶ is a pivolyl, benzoyl or substituted benzoyl protecting group, whereby the substituents are selected from alkyl groups such as methyl, halogen atoms such as Cl, Br, F, and I and NO₂ .

6. The method according to claim 5, further comprising the step of:
selectively protecting the OH groups in 3- and 4-position with a ketal or acetal protecting type protecting group using standard conditions, to yield a compound of general formula (VII): 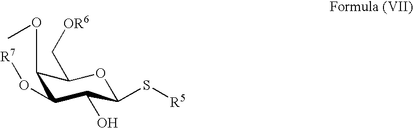
wherein R⁵ represents a straight or branched C₁₋₁₄ alkyl group or a phenyl group optionally substituted with one or more C₁₋₄ alkyl groups and R⁶ is a pivolyl, benzoyl or substituted benzoyl protecting group, whereby the substituents are selected from alkyl groups such as methyl, halogen atoms such as Cl, Br, F, and I and NO₂; and R⁷ represents a ketal or acetal type protecting group selected from the group consisting of benzylidene, 4-nitrobenzylidene, 4-methoxybenzylidene and isopropylidene.

7. The method according to claim 6, further comprising the step of:
protecting the OH group in 2-position of the compound of general formula (VII) as defined in claim 6 with chloroacetyl chloride using standard conditions, to yield a compound of general formula (VIII): 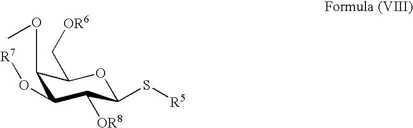
wherein R⁵ represents a straight or branched C₁₋₁₄ alkyl group or a phenyl group optionally substituted with one or more C₁₋₄ alkyl groups;
R⁶ is a pivolyl, benzoyl or substituted benzoyl protecting group, whereby the substituents are selected from alkyl groups such as methyl, halogen atoms such as Cl, Br, F, and I and NO₂ and R⁷ represents a ketal or acetal type protecting group selected from the group consisting of benzylidene, 4-nitrobenzylidene, 4-methoxybenzylidene and isopropylidene; and R⁸ represents a chloroacetyl protecting group.

8. The method according to claim 7, further comprising the step of:
selectively deprotecting the OH group in 3- and 4-position of the compound of general formula (VIII) as defined in claim 7 using standard conditions, to yield a compound of general formula (IX): 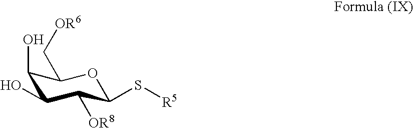
wherein R⁵ represents a straight or branched C₁₋₁₄ alkyl group or a phenyl group optionally substituted with one or more C₁₋₄ alkyl groups; R⁶ is a pivolyl, benzoyl or substituted benzoyl protecting group, whereby the substituents are selected from alkyl groups such as methyl, halogen atoms such as Cl, Br, F, and I and NO₂; and R⁸ represents a chloroacetyl protecting group.

9. The method according to claim 8, further comprising the step of:
reacting the compound of general formula (IX) with a trialkylorthoacetate, benzoate or pivolate to form an 3,4-orthor ester which is subsequently migrated to the axial 4-position under acidic conditions to yield a compound of general formula (X): 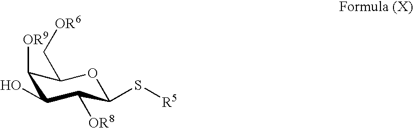
wherein R⁵ represents a straight or branched C₁₋₁₄ alkyl group or a phenyl group optionally substituted with one or more C₁₋₁₄ alkyl groups; R⁶ is a pivolyl, benzoyl or substituted benzoyl protecting group, whereby the substituents are selected from alkyl groups such as methyl, halogen atoms such as Cl, Br, F, and I and NO₂; R⁸ represents a chloroacetyl protecting group; and R⁹ represents a benzoyl, acetyl or pivolyl protecting group.

10. The method according to claim 9, further comprising the step of:
reacting the OH group in 3-position of the compound of general formula (X) as defined in claim 9 with a protected halogen glucose derivative of general formula (XI): 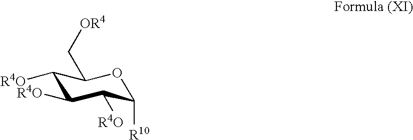
wherein R⁴ independently represents a benzoyl, acetyl or pivolyl protecting group; and R¹⁰ represents a halogen atom, a trichloroacetimidiate group, or a thioalkyl group having 1 to 14 carbon atoms, to yield a compound of general formula (XII): 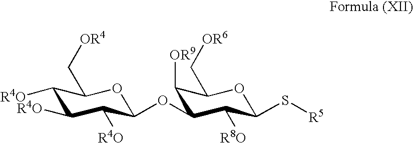
wherein R⁴ independently represents a benzoyl, acetyl or pivolyl protecting group, R⁵ represents a straight or branched C₁₋₁₄ alkyl group or a phenyl group optionally substituted with one or more C₁₋₄ alkyl groups:, R⁶ is a pivolyl, benzoyl or substituted benzoyl protecting group, whereby the substituents are selected from alkyl groups such as methyl, halogen atoms such as Cl, Br, F, and I and NO₂; R⁸ represents a chloroacetyl protecting group; and R⁹ represents a benzoyl, acetyl or pivolyl protecting group.

11. The method according to claim 10, further comprising the step of:
activating the compound of general formula (XII) as defined in claim 10 by oxidizing the thio ether group to a sulfoxide using hydrogen peroxide, and subsequently treating the resulting intermediate with triflic anhydride, to yield a compound of general formula (XIII) 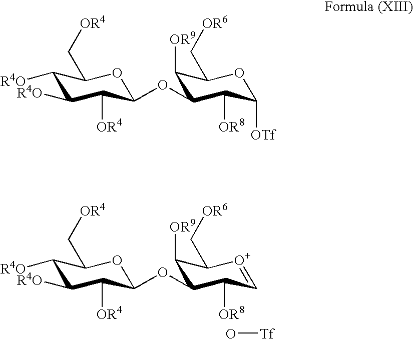
wherein each R⁴ independently represents a benzoyl, acetyl or pivolyl protecting group; R⁶ represents a pivolyl protecting group; R⁸ represents a chloroacetyl protecting group; R⁹ represents a benzoyl, acetyl or pivolyl protecting group; and Tf represents a triflate leaving group.

12. The method according to claim 13, further comprising the step of:
selectively deprotecting the OH group in the 2-position of the compound of general formula (XV) 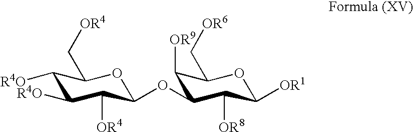
wherein R¹ represents a steroid or a derivative thereof having a hydroxyl group in 3-position and no further unprotected hydroxyl groups; R⁶ represents a pivolyl protecting group; R⁸ represents a chloroacetyl protecting group and R⁹ represents a benzoyl, acetyl or pivolyl protecting group;
using thio urea in the presence of a sterically hindered non-nucleophilic base, and subsequently reacting the resulting intermediate with a protected halogen rhanmose derivative of general formula (XVI): 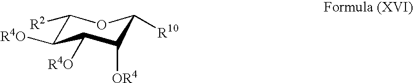
wherein R² represents a straight or branched C₁₋₄ alkyl group or a hydroxyl group; R⁴ independently represents a benzoyl, acetyl or pivolyl protecting group; and R¹⁰ represents a halogen atom, a trichloroacetimidiate group, or a thioalkyl group having 1 to 14 carbon atoms; to yield a compound of general formula (XVII): 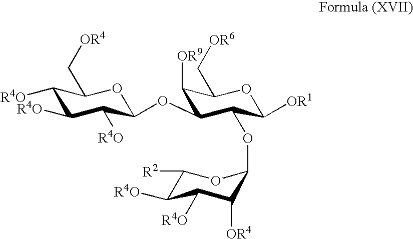
wherein R¹, R², R⁴, R⁶, and R⁹ are as defined above.

13. The method according to claim 12, further comprising the step of:
deprotecting the compound of general formula (XVII) as defined in claim 12, to yield the compound of general formula (I) 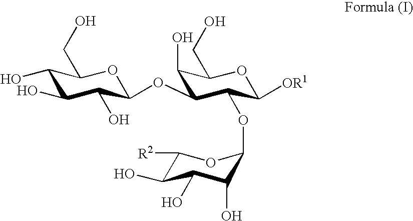
wherein R¹ represents a steroid or a derivative thereof having a hydroxyl group in 3-position and no further unprotected hydroxyl groups; and R² represents a straight or branched C₁₋₄ alkyl group or a hydroxyl group.

14. The method according to claim 1, wherein R¹ represents a tomatidin-3-yl, demissidin-3-yl, solanidin-3-yl and solasodin-3-yl group.

15. The method according to claim 1, wherein R² represents a methyl group.

16. The method according to claim 2, wherein R³ in the compound of general formula (II) represents a bromine atom.

17. The method according to claim 2, wherein R⁴ in the compound of general formula (II) represents an acetyl protecting group.

18. The method according to claim 3, wherein R⁵ in the compound of general formula (III) represents a phenyl group.

19. The method according to claim 6, wherein R⁷ in the compound of general formula (VII) represents a isopropylidene protecting group.

20. The method according to claim 10, wherein R⁴ in the compounds of general formula (XI)represents a benzoyl protecting group.

21. The method according to claim 1, wherein reacting a compound of general formula (XIII) with a compound of general formula (XIV) is carried out in the presence of sterically hindered non-nucleophilic base.

22. The method according to claim 21, wherein the sterically hindered non-nucleophilic base is selected from 2,6-lutidine, 2,4,6-collidine or 2,6-di-tertbutyl-4-methyl pyridine.

23. A steroid modified solatriose of general formula (I) as defined in claim 1, wherein R¹ represents a tomatidin-3-yl or demissidin-3-yl group.

24. A compound of general formula (XVII) as defined in claim 12.

25. A compound of general formula (XV) as defined in claim 1.

26. A compound of general formula (X) as defined in claim 9.

27. A compound of general formula (XII) as defined in claim 10.
